(19) United States
(12) Patent Application Publication
(10) Pub. No.: US 2025/0302904 A1
(43) Pub. Date: Oct. 2, 2025

(54) EXTRACTS IN EUTECTIC SOLVENT OF OLIVE OIL POLYPHENOLS, COMPOSITIONS, USES AND METHODS OF PREPARATION THEREOF

(71) Applicant: ACTIVE-ITALIA S.R.L., Rome (IT)

(72) Inventors: Rodolfo Federico, Rome (IT); Luciana Mosca, Rome (IT); Antonio Francioso, Rome (IT); Roberto Mattioli, Rome (IT); Eleonora Miceli, Grottaferrata RM (IT); Umberto Berti, Rome (IT)

(73) Assignee: ACTIVE-ITALIA S.R.L., Rome (IT)

(21) Appl. No.: 18/864,406

(22) PCT Filed: May 11, 2023
(86) PCT No.: PCT/IB2023/054868

§ 371 Date: Nov. 8, 2024

(30) Foreign Application Priority Data
May 11, 2022 (IT) 102022000009722

(51) Int. Cl. A61K 36/63; A61K 9/00

(52) U.S. Cl. CPC A61K 36/63; A61K 9/0014

(57) ABSTRACT

The present invention relates to an extract in eutectic solvent of olive oil polyphenols, as well as compositions or medical devices comprising it, in particular for use in medicine. The invention also relates to a process for the preparation of said polyphenolic extract.

### FIELD OF THE INVENTION

The present invention relates to an extract in eutectic solvent of olive oil polyphenols, as well as pharmaceutical compositions or medical devices including it, in particular for use in medicine. The invention also relates to a process for the preparation of said extract.

### STATE OF ART

Skin is the organ with largest sizes of the human being and it represents the first defensive barrier against external attacks such as pathogens, solar rays, chemical agents, exposure to high or low temperature and dehydration. The epithelium integrity is fundamental so that this could exert its barrier function; however, many stresses to which one is exposed on a daily basis can compromise its functionality. This can translate into the occurrence of dermatological diseases which can lead to inflammatory manifestations and immune disorders, exacerbated by underlying genetic pathologies. Among the most common disorders there are: atopic dermatitis, seborrheic dermatitis, psoriasis, acne, rosacea, contact dermatitis, couperose, allergic dermatitis, keloids.

The dermatological pathologies often represent a considerable discomfort for the patient from a psychological point of view; moreover, currently there is no resolutive cure for most of these diseases. Most products widely used for the treatment of the dermatological diseases do not represent a definitive solution to the problem since often such pathologies are associated to inflammation and disorders of the immune system.

Therapeutic agents containing active principles aimed at solving only one of the pathology aspects cannot have a role as curative agents since they do not act on all contributing causes. First drugs for the treatment of these pathologies often are cortisone-based drugs or vitamin D derivatives. The cortisone-based drugs are the most used ones and they are widely used to hinder the inflammatory processes; however, their long-term effectiveness is limited and the prolonged use can cause side effects going from skin atrophy to the occurrence of viral or bacterial infections since they suppress the physiological immune response. Some of these treatments can cause skin irritation and they cannot be used in the long run. The fact that, upon suspending the treatment, the pathology can recur with even more severity is particularly serious. Monoclonal antibodies are currently in use for particularly serious forms of the disease, but they are not free from side effects (that is occurrence of some cancer forms) apart from having a very high cost.

Therefore, the detection of molecules which are capable of exerting anti-inflammatory effects without suppressing the immune response, on the contrary by stimulating it, represents one of the main challenges of the field. In such context, the polyphenols, a wide class of molecules isolated from several plants, represent one among the most interesting options.

Among the therapeutic polyphenols with anti-inflammatory, antitumoral, immunomodulating, neuroprotective and cardioprotective activity, the polyphenols extracted from oil olive attracted particular attention. These molecules form during the milling and kneading process characteristic of the olive oil production process, and they are present nor in the origin plant (Olea europea) nor in the fruits thereof (drupe). Among the polyphenolic compounds present in the oil it is possible to find: simple phenols (tyrosol, hydroxytyrosol), secoiridoids (oleocanthal, oleacein) and the respective glycosides (ligstroside, oleuropein), flavonoids and lignans (apigenin, luteolin and pinoresinol).

Different methods to extract and concentrate the polyphenols starting from olive oil are known in the field. However, all these methods use organic solvents such as methanol and acetonitrile, and they use hexane as delipidizing agent with the purpose of removing possible contaminations from triglycerides. Therefore, the application of such methods mainly involves the use of substances potentially toxic for the operator and surely dangerous for the environment. Moreover, such methods require that the used olive oil treated with organic solvents is disposed of by following specific procedures dedicated to the toxic substances.

In such context, then, the need appears to be still very urgent for having available compositions based on natural polyphenols effective for the treatment of dermatological pathologies and obtainable through effective and at the same time economically advantageous and sustainable methods.

### SUMMARY OF THE INVENTION

The objective underlying the present invention is to provide new extracts based on olive oil polyphenols provided with anti-inflammatory, anti-oxidant and immuno-modulating activity allowing to meet the needs mentioned above with reference to the known art. The present invention is based upon the finding that the use of eutectic solvents, and in particular Natural Deep Eutectic Solvents (NaDES), allows to improve the clinical effectiveness of the action of polyphenols and allows a stabilization thereof, by allowing to prolong the shelf-life of the so-obtained products.

Therefore, the invention relates to an extract in eutectic solvent of polyphenols derived from olive oil as well as a process for the production of such extract of polyphenols comprising the following steps of:
(i) providing an olive oil comprising said polyphenols;
(ii) putting in contact said olive oil with at least one eutectic solvent for a period of time sufficient to allow the extraction of said polyphenols in said at least one eutectic solvent;
(iii) separating the extract of polyphenols in eutectic solvent obtained in step (ii) from olive oil.

The main advantage of the process, the invention relates to, lies in the use of solvents comprising molecules of natural origin, biocompatible, not toxic, free of components of synthetic origin. The solvents usable in the process of the invention, in fact, mainly consist of environmentally sustainable molecules and, unlike many organic solvents, they do not damage the environment and do not require to take particular precautions or disposal protocols.

The use of natural eutectic solvents for the preparation of the polyphenolic extracts, the present invention relates to, further allows to solve some technical problems resulting from use of polyphenols and to exert unexpected therapeutic effects.

Firstly, the use of eutectic solvents allows to obtain, in one single extracting step, an extract highly enriched with olive oil polyphenols, by avoiding the use of toxic organic solvents. Advantageously, the so obtained extracts can be directly used in the applications of interest or in the formulation of finished compositions, without the need for applying additional purification steps. The extractive process of the invention in this way results to be much cheaper than the classical extractive techniques, which generally require the application of subsequent steps of chromatographic purification.

Secondly, the use of eutectic solvents, in particular NaDES, allows to obtain a stable polyphenolic extract. In other terms, the shelf-life of the polyphenolic extract is guaranteed by the stabilizing effect of the eutectic solvent, which prevents oxidation and degradation of polyphenols. The eutectic solvent in fact allows to entrap the polyphenolic compounds in a cage of hydrogen bonds, by hindering the onset of degradative processes caused by humidity, heat or oxidative and reaction processes with other molecules, by strengthening the pharmacological effectiveness thereof. This is particularly useful in order to preserve the stability of the most important active principles among the polyphenols present in the extracts, oleocanthal and oleacein, which, since they are two dialdehydes, are subjected to rapid degradation and quick reactivity in presence of amino substances with formation of a stable Shiff base.

Apart from what illustrated above, the authors of the present invention have surprisingly found that the molecules constituting the eutectic solvent used in the extracting process show a synergic action in presence of the polyphenols, by enhancing the biological activity thereof. In particular, betaine, a compound with quaternary ammonium used in some of the eutectic solvents present in the extracts this invention relates to, is provided with anti-inflammatory activity with mechanism different from that of the polyphenols; nevertheless, it was observed that such compounds result to be capable of enhancing the anti-inflammatory capabilities of the olive oil polyphenols.

Surprisingly, the synergetic effect observed by the authors of the invention results to be stable even in the long-run. As it is deducible from the experimental results illustrated in FIGS. 1-3, the extracts and compositions devised by the authors of the invention exert a very precocious powerful effect, with a very rapid onset response, within very few hours and comparable to that of a powerful cortisone-based drug. Such effects are kept in the long run without showing any side effect. In the long run immunomodulating and analgesic effects were also highlighted. Effects of so rapid onset and protracted effect are unusual and not documented in literature.

Thanks to the above-illustrated advantages the method, the present invention relates to, can be used in several therapeutic applications such as in particular, the prevention and/or treatment of different skin pathologies or disorders, for example psoriasis, atopic dermatitis or allergic dermatitis. Therefore, the invention also relates to:
a composition for topical use comprising an extract according to any one of the herein described embodiments and at least a pharmaceutically acceptable carrier or excipient;
a medical device coated or soaked with an extract or with a composition according to any one of the herein described embodiments;
an extract, a composition for topical use or a device according to any one of the herein described embodiments for use in medicine, in particular for use in the treatment or in assisting the treatment of a skin pathology or disorder; and
the use of a eutectic solvent according to any one of the embodiments described in the present invention for extracting polyphenols from olive oil.

Other advantages and features of the present invention will result evident from the following detailed description.

### BRIEF DESCRIPTION OF FIGURES

FIG. 1. Serum application according to an embodiment of the invention as described in Example 4 on a hand suffering from atopic dermatitis resistant to treatment with cortisone-based drugs. The photos were taken immediately before the application, after 10 hours, after 32 hours and after 7 days. The improvements start to be evident already as from 32 hours after the start of treatment.

FIG. 2. Serum application according to an embodiment of the invention as described in Example 4 on the scalp of a patient suffering from psoriasis. The photos were taken immediately before, after 1 day, after 3 days and after 10 days of treatment. The serum effect appears by decreasing the affected area and reducing the level of redness of the involved portion.

FIG. 3. Application of the cream described in Example 6 on the face skin suffering from allergic dermatitis. The photos were taken immediately before the application and after 1 day where a marked improvement in symptoms is observed.

FIG. 4. Stability over time (6 months) of oleacein and oleocanthal at 25° C. and 40° C. in water and in the formulated product (serum NaDES diluted in almond oil). The values represent standard averages and deviations (N=3). The significance tests were performed with one-way ANOVA (p<=0.05).

FIG. 5. Effect of application of polyphenols extracted in NaDES diluted in almond oil, according to an embodiment of the invention as described in Example 12, on permanent lesions resistant to treatment with cortisone-based drugs, in particular plaque psoriasis. The reduction in the level of redness of the involved area and the disappearance of plaques and raised areas are observed.

FIG. 6. Effect of application of polyphenols extracted in NaDES diluted in almond oil, according to an embodiment of the invention as described in Example 12, on permanent lesions resistant to treatment with cortisone-based drugs. A reduction in the suffering area and an enormous decrease in the level of redness of the area after 10, 20 and 30 days of treatment are observed. The desquamation phenomena too result to be reduced.

FIG. 7. Effect of application of polyphenols extracted in NaDES diluted in almond oil, according to an embodiment of the invention as described in Example 12, on permanent lesions resistant to treatment with cortisone-based drugs, in particular palmoplantar psoriasis. Complete disappearance of the lesions with reduction in the desquamation phenomena after 5 days of treatment.

FIG. 8. Effect of application of polyphenols extracted in NaDES diluted in almond oil, according to an embodiment of the invention as described in Example 13, on a three-month wound following surgical operation after 15 days of treatment.

FIG. 9. Effect of application of polyphenols extracted in NaDES diluted in almond oil, according to an embodiment of the invention as described in Example 13, on three-year keloid following nevus removal. A reduction in the thickening and hardness of keloid after 15 days of treatment is observed. The redness too appears decreased.

FIG. 10. Effect of application of polyphenols extracted in NaDES diluted in almond oil, according to an embodiment of the invention as described in Example 13, on four-year keloid following surgical operation. A reduction in the thickening and hardness of the keloid after 30 days of application is observed. The redness too appears decreased.

FIG. 11. Effect of application of polyphenols extracted in NaDES diluted in almond oil, according to an embodiment of the invention as described in Example 14, on iron burn. The almost total disappearance of the lesion after 10 days of treatment is highlighted.

FIG. 12. Results of a subject treatment with anti-aging cream according to an embodiment of the invention as described in Example 15, for 45 days. The marked reduction in wrinkles, spots and UV damage is highlighted, apart from a volumetric increase of the malar area comparable to 1 ml of volumizing filler apart from a lifting of the whole middle third of the face appreciable from the opening of the nasogenial wrinkle symptomatic of an effective biorestructuring of the overlying connective tissue.

### GLOSSARY

The terms used in the present description are as generally comprised by the person skilled in the art, unless otherwise indicated.

Under the term “eutectic solvent” or “deep eutectic solvent” (DES), in the context of the present invention, a solvent consisting of two or more components is meant, in particular at least a hydrogen bond acceptor (HBA) and a hydrogen bond donor (HBD) interacting therebetween by associating and forming a eutectic mixture with a melting temperature much lower than that of its components. It is assumed that the self-association occurs through interactions of hydrogen bond, even if it is assumed that even Van der Waals forces can play a relevant role.

The DES mentioned in the present description and claims are known in the field even as “low transition temperature mixtures” (LTTM).

Differently from the conventional solvents, such as ethanol, methanol and so on, the deep eutectic solvents are not volatile, which means that they have a very low vapour pressure and then they are hardly flammable. The toxicity of the eutectic solvents is very low, their biodegradability is high and the precursors required for their production are generally cheap, easily and abundantly available and renewable.

Under the term “natural deep eutectic solvent”, NaDES in short, in the context of the invention, a eutectic solvent is meant obtainable starting from precursors coming from natural sources, such as for example, carbohydrates, organic acid, polyols, sugars or amino acids.

In the context of the present invention, the term “betaine” relates to the N,N, N-trimethylglycine compound, unless otherwise indicated.

In the context of the invention, under the expression “extract in eutectic solvent” an extract of polyphenols or derivatives from olive oil is meant, obtained by using a eutectic solvent according to any one of the herein described embodiments as extraction solvent. According to a preferred aspect of the invention, the expression “extract in eutectic solvent” specifically relates to an extract of olive oil polyphenols or derivatives by using a eutectic solvent as extraction solvent by means of a process according to any one of the embodiments as defined in the present description and claims.

In any point of the present description and claims, under the term propylene glycol or propyleneglycol the 1,2-propanediol compound is meant, also known as a-propylene glycol.

In any point of the present description and claims, the term 1,3-propanediol relates to the isomer of the propylene glycol also known as β-propylenglycol.

In any point of the present description and claims, the term “comprising” can be replaced by “consisting of”.

### DETAILED DESCRIPTION OF THE INVENTION

As mentioned, a first aspect of the present invention is represented by an extract in eutectic solvent of polyphenols or derivatives from olive oil.

Preferably, said at least eutectic solvent is any natural deep eutectic solvent (NaDES) known in the field.

A eutectic solvent suitable to be used as extraction solvent for preparing an extract according to any one of the herein described embodiments is a solvent stable and transparent at room temperature, that is a solvent which can be kept for long periods at room temperature, without encountering appreciable aggregation or crystallization phenomena.

Such solvent can include two or more compounds selected from hydrogen bond acceptor compounds and hydrogen bond donor compounds known in the field. Said eutectic solvent can include two, three, four, five, six, seven, eight, nine or more components which can be selected from hydrogen bond acceptor compounds and hydrogen bond donor compounds known in the field, preferably two or three components only.

According to a particular aspect of the present invention, said eutectic solvent comprises at least a hydrogen bond acceptor compound selected in the group consisting of choline, betaine, proline, acetylcholine, urea, thiourea, methyl urea, dimethyl urea, trifluoromethyl urea, carnitine, ethyl ammonium chloride, diethyl ammonium chloride, acetamide, trifluoroacetamide and salts or derivatives thereof, and at least a hydrogen bond donor compound selected in the group consisting of sugars (in particular glucose, fructose, saccharose, maltose, dextrose, ribose, galactose), amino acids (in particular proline, serine, glycine, alanine, glutamic acid), carboxylic acids (in particular malic acid, lactic acid, tartaric acid, benzoic acid, itaconic acid, citric acid, isocitric acid, caffeic acid, adipic acid), polyols (in particular glycerol, 1,2-propanediol, 1,3 propanediol, inositol, butanediol, ethylene glycol, triethylene glycol, sorbitol, xylitol, ribitol, erytritol, maltitol, mannitol and lactitol) and ammine and salts or derivatives thereof.

Not limiting examples of eutectic solvents usable as extraction solvent for preparing an extract according to any one of the herein described embodiments comprise a hydrogen bond donor compound selected from glycerol, propylene glycol, benzoic acid, citric acid, succinic acid, urea, benzamide and any one of the compounds listed in the present description and salts or derivatives thereof.

According to a preferred aspect of the invention, said at least a eutectic solvent comprises at least a hydrogen bond acceptor compound selected in the group consisting of choline, in particular choline chloride or choline acetate, betaine, proline, carnitine and derivatives thereof, and at least a hydrogen bond donor compound selected in the group consisting of glycerol, propylene glycol, ethylene glycol.

In a preferred embodiment of a method according to the present invention, said eutectic solvent is a binary mixture consisting of a hydrogen bond acceptor compound and a hydrogen bond donor compound according to any one of the herein described embodiments.

Preferably, said eutectic solvent is a NaDES comprising or consisting of propylene glycol and betaine, or propylene glycol and choline, or propylene glycol and proline.

Preferably, said eutectic solvent is a NaDES comprising or consisting of 1,3-propanediol and betaine, or 1,3-propanediol and choline, or 1,3-propanediol and proline.

Alternatively, said eutectic solvent is a NaDES comprising or consisting of glycerol and betaine, or glycerol and choline, or glycerol and proline.

According to an aspect of the invention, said at least a hydrogen bond acceptor compound and said at least a hydrogen bond donor compound are present in said eutectic solvent in a molar ratio comprised between 10:1 and 1:10. In particular, said eutectic solvent can include at least a hydrogen bond acceptor compound and at least a hydrogen bond donor compound in a molar ratio equal to: 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10 (mol: mol), preferably in a molar ratio equal to 1:1.5 or 1:1.8 o 1:2.2 or 1:3 or 1:3.2, 1:3.3, or 1:4.

According to a preferred aspect of the invention, said eutectic solvent comprises betaine and propylene glycol in a molar ratio equal to 1:3.3 or comprises betaine and glycerol in a molar ratio equal to 1:2,2, or comprises betaine and 1,3-propanediol in a molar ratio equal to 1:3.3.

According to an additional aspect of the invention, said eutectic solvent comprises choline and propylene glycol in a molar ratio equal to 1:3 or comprises choline and glycerol in a molar ratio equal to 1:1,5, or comprises choline and 1,3-propanediol in a molar ratio equal to 1:3.

According to an additional aspect of the invention, said eutectic solvent comprises proline and propylene glycol in a molar ratio equal to 1:4 or comprises proline and glycerol in a molar ratio equal to 1:1,8, or comprises proline and 1,3-propanediol in a molar ratio equal to 1:3.

In a variant according to the present invention, said eutectic solvent can include three components which can be selected among hydrogen bond donor and acceptor compounds according to any one of the embodiments known to a person skilled in the field such as, for example, the compounds defined in the present description and claims; in particular, said three components can be present in the eutectic solvent in a molar ratio comprised between 10:1:1 and 1:1:10.

Preferably, said eutectic solvent comprises choline chloride, betaine and propylene glycol in molar ratio equal to 1:1:3 up to 1:1:10, preferably 1:1:6,3.

Alternatively, said eutectic solvent comprises choline chloride, betaine and 1,3-propanediol in molar ratio equal to 1:1:3 up to 1:1:10, preferably 1:1:6,3.

Depending upon the selected precursors, for example selected hydrogen bond acceptors or donors, a person skilled in the field will be capable of dosing the amounts and molar ratios of such compounds with the purpose of obtaining a eutectic solvent stable and transparent at room temperature, that is a eutectic solvent preservable for long periods at room temperature, in absence of aggregation or crystallization.

According to an additional aspect of the invention, the eutectic solvent used as extraction solvent for preparing an extract according to any one of the herein described embodiments can be a mixture of two or more eutectic solvents, which could be selected by a person skilled in the field based upon their compatibility.

A eutectic solvent suitable to be used to obtain an extract according to the present invention can be used by using any one of the techniques known to a person skilled in the field, for example any one of the methods as illustrated hereinafter in the present description or claims.

As already mentioned, the polyphenols present in the extract according to any one of the embodiments of the invention are polyphenols of natural origin and, in particular, polyphenols or derivatives from olive oil. Preferably, the olive oil is produced by olives obtained from plants of the Olea europaea species, such as Leccino, Itrana, Taggiasca, Olivotto, Quercetana, Pendolino, Orbetana, Nocellara, Ortice, Ravese, Marinese, Biancolilla, Arbequina, Hojiblanca, Lechín, Manzanilla, Koroneiki, Coratina, Picual and/or Verdial.

Still more preferably, the olive oil is obtained from a cultivar of Olea europaea particularly enriched with polyphenols, such as Coratina, Frantoio, Ogliarola, by using any one of the techniques known to a person skilled in the field. According to an aspect of the invention, said olive oil is virgin olive oil or extra-virgin olive oil.

In an embodiment, the polyphenols present in an extract according to the invention comprise one or more compounds which can be selected from simple phenols (tyrosol, hydroxytyrosol), secoiridoids (oleocanthal, oleacein) and the respective glycosides (ligstroside, oleuropein), flavonoids and lignans (apigenin, luteolin and pinoresinol), or mixtures thereof.

In a preferred embodiment, the polyphenols present in an extract according to the invention comprise oleocanthal and/or oleacein. Oleocanthal is a natural compound present in virgin olive oils. It belongs to the family of polyphenols. The importance and peculiarity of oleocanthal lies in the fact of having antioxidant and anti-inflammatory features. Its anti-inflammatory action on the organism is very similar to ibuprofen, one of the most taken non-steroidal anti-inflammatory drugs. Moreover, its activity as platelet aggregation inhibitor was noted.

Oleacein, another olive oil polyphenol, showed properties very similar to those of oleocanthal and, in particular, it has an optimum antioxidant and antiproliferative activity.

The polyphenols present in an extract in eutectic solvent according to the present invention are stabilized by the presence of the eutectic solvent. According to an aspect of the invention, said polyphenols are present in the extract in an amount from 1 to 200 mg/mL. The evaluation of the content of polyphenols in the extract can be performed both by means of a colorimetric method (for example Folin Ciocalteau) and by chromatographic analysis (for example UHPLC in reverse phase with spectrophotometric and mass detector).

In a preferred aspect of the invention, an extract in eutectic solvent according to any one of the herein described embodiments can be obtained through a process according to any one of the embodiments described in the present application.

### Preparation Processes and Uses

The present invention also relates to a process for preparing an extract of polyphenols or derivatives from olive oil, comprising the following steps of:
(i) providing an olive oil comprising said polyphenols;
(ii) putting in contact said olive oil with at least one eutectic solvent for a period of time sufficient to allow the extraction of said polyphenols in said at least one eutectic solvent;
(iii) separating the extract of polyphenols in eutectic solvent obtained in step (ii) from olive oil.

A eutectic solvent suitable to be used in a process according to the present invention is a eutectic solvent according to any one of the embodiments as defined in the present description and claims.

In a preferred embodiment of the process, the used eutectic solvent is a NaDES comprising or consisting of propylene glycol and betaine, or propylene glycol and choline, or propylene glycol and proline, or 1,3-propanediol and betaine, or 1,3-propanediol and choline, or 1,3-propanediol and proline. Alternatively, said eutectic solvent is a NaDES comprising or consisting of glycerol and betaine, or glycerol and choline, or glycerol and proline. The above-mentioned components can be present in the eutectic solvent in any one of the molar ratios as previously defined in the text of the present description.

The olive oil used in a process according to the present invention is an olive oil according to any one of the previously described variants, preferably it is an oil obtained from a cultivar of olives enriched with polyphenols, such as Coratina, Frantoio, Ogliarola, still more preferably it is an extra-virgin olive oil.

In process step (ii), said olive oil and said eutectic solvent are preferably put in contact or mixed in a ratio from 100:1 to 1:100, still more preferably equal to 20:1.

Advantageously, step (ii) and/or (iii) of a method according to the herein described embodiments, can be performed at room temperature, preferably at a temperature comprised between 2° and 30° C.

According to an aspect of the invention, step (ii) has a duration comprised between 2 and 60 minutes, preferably at least equal to 30 minutes.

In another embodiment, the process of the invention can further include the following step, preceding said step i), of:
i-0) mixing at least a hydrogen bond acceptor compound and at least a hydrogen bond donor compound as defined in the present description and/or claims so as to obtain said at least a eutectic solvent.

Step i-0) can be performed by mixing, optionally under stirring, by using any one of the techniques known to a person skilled in the field. In particular, step i-0) can be performed at a temperature comprised between 6° and 90° C., preferably at a temperature equal to 80° C. According to an aspect of the invention, step i-0) has a duration comprised between 5 and 120 minutes, preferably it has a duration equal to 30 minutes.

The process step (iii) can be performed by using any one of the techniques known to a person skilled in the field which could allow an adequate phase separation between the used extraction solvent (that is the eutectic solvent) and the olive oil and then an easy recovery of the extraction solvent; preferably, step (iii) can be performed by decantation and/or centrifugation. After decantation and/or centrifugation of the mixture of olive oil and eutectic solvent, the eutectic solvent comprising the extracted polyphenols could be suitably recovered by any one of the known techniques.

By pure way of example, the centrifugation of the mixture olive oil/eutectic solvent can be performed at 500 g for 20 minutes.

An additional aspect of the invention relates to an extract of olive oil polyphenols in eutectic solvent obtainable by means of a process according to any one of the herein described embodiments.

The present invention also relates to the use of a eutectic solvent for extracting polyphenols from olive oil, or for preparing an extract of polyphenols starting from olive oil.

According to an aspect of the invention, said eutectic solvent is any eutectic solvent known to a person skilled in the field, in particular a eutectic solvent according to any one of the embodiments as defined in the present description and claims.

In a preferred embodiment, said eutectic solvent is a NaDES, in particular it is a NaDES comprising the combination of at least a hydrogen bond donor compound and at least a hydrogen bond acceptor compound according to any one of the previously described variants.

A preferred embodiment of the invention relates to the use of a NaDES for extracting polyphenols from olive oil, wherein said eutectic solvent comprises at least a hydrogen bond acceptor compound selected in the group consisting of choline chloride, choline acetate, betaine, proline, carnitine and derivatives thereof, and at least a hydrogen bond donor compound selected in the group consisting of propylene glycol, 1,3-propanediol, glycerol, ethylene glycol.

According to an aspect of the invention, said eutectic solvent comprises at least a hydrogen bond acceptor compound and at least a hydrogen bond donor compound in a molar ratio according to any one of the embodiments described in the present application.

In a preferred embodiment of a use according to the present invention, said eutectic solvent is a binary mixture consisting of a hydrogen bond acceptor compound and a hydrogen bond donor compound according to any one of the previously described embodiments. Preferably, said eutectic solvent is a NaDES comprising or consisting of propylene glycol and a compound selected among choline chloride and betaine, or is a NaDES comprising or consisting of propylene glycol, choline chloride and betaine, or it is a NaDES comprising or consisting of 1,3-propanediol and a compound selected among choline chloride and betaine, or it is a NaDES comprising o consisting of 1,3-propanediol, choline chloride and betaine. The above-mentioned components can be present in the eutectic solvent in any one of the molar ratios as previously defined in the text of the present description.

The use according to any one of the herein described embodiments can be performed or implemented through a process or one or more process steps according to any one of the embodiments illustrated in the present description and claims.

A process or use according to any one of the embodiments described in the present invention description and claims can further include one or more analytical characterization steps aimed at determining the amount of polyphenols present in the extract in obtained eutectic solvent, for example by using any one of the techniques known to a person skilled in the field, such as colorimetric analysis or chromatography.

### Pharmaceutical Compositions and Medical Devices

The present invention further relates to a composition for topical use comprising an extract according to any one of the embodiments illustrated in the present description and claims and at least a pharmaceutically acceptable excipient or carrier, in particular at least a dermatologically acceptable excipient or carrier.

According to an aspect of the invention, the composition includes an extract according to any one of the herein described embodiments in an amount at least equal to 0.5%, 1%, 2%, 5%, 10%, 20%, 30%, 40%, 50%, 60%, 70%, 80%, 90%, or 99% by weight with respect to the total weight of the composition.

Pharmaceutically acceptable carriers or excipients are well known in the art and they could be selected based upon the final use of the composition. Possible additional components required for the formulation of such compositions can be selected by the person skilled in the field based upon the known art.

An additional embodiment of the invention provides that the composition could include even olive oil and/or almond oil or any other oil useful in the cosmetic field, such as argan oil, linseed oil, sunflower oil, wheat germ oil, rice bran oil, coconut oil, hemp oil, baobab oil, avocado oil, St. John's wort oil, jojoba oil, macassar oil, pomegranate oil, neem oil, rosehip oil, pumpkin seed oil, sesame oil, grapeseed oil, including oils enriched with omega-6 and omega-3.

As mentioned, the compositions according to any one of the herein described embodiments are preferably for topical use and they can be formulated in one of the following forms: serum, cream, ointment, oil bath, shampoo, spray, pasta, hydrophilic gel, hydrophobic gel, foam, emulsion, suspension or solution. Depending upon the selected formulation, a person skilled in the field could select suitable excipients and/or carriers known in the field suitable to be used in the composition.

According to a preferred aspect of the invention, the composition is formulated in form of serum or cream. By pure way of example, a composition in form of cream according to any one of the herein described embodiments could include, as additional excipients or carriers one or more of the following compounds, preferably all following compounds: water, cetearyl alcohol, Simmondsia chinensis oil, glycerine, Oryza sativa bran oil, squalane, Aloe barbadensis leaf juice, glyceryl stearate, filtered extract of Pseudozyma epicola/Camellia japonica seed oil ferment, olive glyceryl, alkane C13-14, polygliceryl-4-oleate, cetearyl glucoside, panthenol, perfume, potassium sorbate, sodium benzoate, tocopheryl acetate, xanthan gum, Mel extract, tetrasodium glutamate diacetate, lactic acid, glucose, sodium hyaluronate, phenoxyethanol and ethylhexylglycerin.

A preferred aspect of the invention relates to a composition for topical use comprising an extract according to any one of the herein described variants, in particular an extract comprising a NaDES based on betaine/propyleneglycol or a NaDES based on betaine/1,3-propanediol, and a preparation in cream comprising water, cetearylic alcohol, Simmondsia chinensis oil, glycerine, Oryza sativa bran oil, squalane, Aloe barbadensis leaf juice, glyceryl stearate, filtered extract of Pseudozyma epicola/Camellia japonica seed oil ferment, olive glyceryl, alkane C13-14, polygliceryl-4-oleate, cetearyl glucoside, panthenol, perfume, potassium sorbate, sodium benzoate, tocopheryl acetate, xanthan gum, Mel extract, tetrasodium glutamate diacetate, lactic acid, glucose, sodium hyaluronate, phenoxyethanol and ethylhexylglycerin. Preferably, said extract and said preparation in form of cream are mixed in ratio from 100:1 to 1:100 by using conventional pharmaceutical techniques.

An embodiment according to the invention in particular relates to a composition in form of serum comprising an extract according to any one of the herein described variants, in particular an extract comprising a NaDES based on betaine/propyleneglycol or a NaDES based on betaine/1,3-propanediol, and sweet almond oil or olive oil. Preferably, in the serum, said extract is mixed with sweet almond oil or olive oil in ratio 10:1 to 1:10. Mixing can be performed by usual pharmaceutical technique which guarantees the formation of a fine emulsion.

An additional embodiment relates to a composition comprising an extract according to any one of the herein described variants and almond oil, in particular at least two portions by volume of an extract according to any one of the herein described variants and one portion by volume of almond oil.

All herein described embodiments could even comprise hydrating substances such as collagen, elastin, various protein hydrolysates, hydrolysates of nucleic acids, natural hydrating factors, absorption promoters, fatty acids, fragrances, essences and/or preservatives.

The compositions according to any one of the embodiments illustrated in the present description and claims can be prepared by using techniques known to a person skilled in the field, comprising for example at least a step of mixing a suitable amount of an extract according to the invention with one or more pharmaceutically suitable excipients and/or carriers.

According to an aspect of the invention, the compositions described in the present application are pharmaceutical compositions.

The invention also relates to a product, medicament or medical device comprising an extract or a composition according to any one of the herein described embodiments. A preferred embodiment of the invention in particular relates to a medical device coated or soaked with an extract or with a composition according to any one of the herein described embodiments

According to a preferred aspect, said product or device is represented by a tissue coated, covered, soaked or impregnated with an extract or with a composition according to any one of the herein described embodiments. Said tissue for example can be represented by a gauze, plaster, bandage, cloth or non-woven fabric.

### Medical Uses

As mentioned above, the authors of the present invention have found that an extract in eutectic solvent of olive oil polyphenols represents a solution with high therapeutic effectiveness for the treatment of several disorders and pathologies.

Therefore, an aspect of the invention relates to an extract, a composition including it, or a product or medical device according to any one of the herein described embodiments for use in medicine or for use as medicament.

According to the present invention an extract or a pharmaceutical composition including it as defined in the present description and claims can be used in particular for the prevention, therapeutic treatment or to assist the therapeutic treatment of skin pathologies or disorders, in particular diseases associated to pain, for example caused by trauma.

An extract or a composition according to the present invention can be used for example to promote healing and/or to reduce the inflammatory processes of the injured skin.

An extract or a composition or a device according to the present invention can be also used to prevent or treat the skin radiation damage, in particular the skin damage caused by exposure to sunlight or to radiotherapy and/or radio-chemiotherapy.

Preferred examples of skin pathologies, disorders or diseases which can take advantage from a treatment with an extract or a composition or device including it according to the present invention comprise psoriasis, in particular plaque psoriasis or palmoplantar psoriasis, atopic dermatitis, seborrheic dermatitis, acne rosacea, allergic dermatitis, skin fibrotic lesions such as keloids, in particular keloids deriving from surgical operation, skin irritations, and skin redness caused or deriving from exposure to sunlight or skin treatments such as making tattoos.

An extract, a composition or device according to the present invention can also be used for the prevention, treatment or to assist the treatment of skin lesions, in particular skin lesions selected among scalding, burns, scalds, insect stings, radiation lesions, in particular lesions caused or deriving from exposure to sunlight or radiotherapy and/or radio-chemiotherapy.

A preferred aspect of the invention relates to an extract or composition according to any one of the herein described embodiments for use in the prevention, in the treatment or to assist the treatment of psoriasis, atopic dermatitis and/or allergic dermatitis.

According to an additional aspect, an extract or a composition according to any one of the herein described variants can be used for the prevention, for the treatment or to assist the treatment of pathologies caused by and/or associated to inflammations and/or pain, for example chronic pain. In particular, said extracts or compositions can be used for the prevention, the treatment or to assist the treatment of pathologies wherein inflammation and immune disorders combine, such as: rheumatoid arthritis, inflammatory bowel diseases, multiple sclerosis, or systemic erythematosus lupus.

A preferred embodiment of the invention in particular relates to an extract or a composition according to any one of the herein described variants for the prevention, the treatment and/or to assist the treatment of chronic pain associated to psoriatic arthritis.

In an embodiment, a treatment which provides the use of an extract or a composition according to the invention can be performed in association with other drugs, such as drugs conventionally used for the treatment of skin pathologies or disorders or of any one of the previously mentioned pathologies, at dosages therapeutically effective for a subject requiring them.

The present invention also relates to the cosmetic use of an extract or a composition according to any one of the herein described embodiments for the treatment and/or prevention of imperfections of a subject skin, in particular of a healthy subject. Preferably, an extract or composition according to any one of the herein described embodiments can be used to prevent, reduce and/or remove the skin wrinkles or aging. Advantageously, an extract or composition according to the present invention can be used as anti-aging cosmetic agents or products.

According to an aspect of the invention, an extract or a composition according to any one of the herein described embodiments can be administered by topical route, optionally in combination to one or more additional active principles, to a subject requiring them.

Preferably, the extract or composition as defined in the present description and claims could be administered by topical route in one or more daily doses.

A pharmaceutical composition according to the invention could be administered for example in an a therapeutically effective amount to a subject requiring them according to any one of the modes for administering a composition in topical form known to a person skilled in the field, such as those previously exemplified in the present description.

According to a preferred aspect of the invention, the extract or a composition as described in the present description and claims could be administered to a subject requiring them by topical route within a base cream. A base cream suitable to be used for a topical application of an extract or a composition according to the invention, for example, is a hydrophilic base cream, that is an oil neutral emulsion in water, suitable as carrier of the extract as such to ease the spreading of the active principles present therein on the skin. Said cream could include one or more dermatologically acceptable carriers or excipients known to a person skilled in the field, such as for example those exemplified in the present description. According to an aspect of the invention, one or more portions of the extract or composition according to the invention could be dissolved within a base cream so as to provide a therapeutically effective dosage for the treatment of any one of the previously mentioned pathologies or disorders.

An extract or a composition according to the invention could be applied directly on the area involved by the pathology at the skin level, preferably several times a day.

The definition of the optimum dosage of said extract or composition according to the invention could vary based on the selected administration form as well as depending upon the clinical picture of the subject subjected to treatment (for example even based upon age, body weight, gender of the subject) or in particular based upon the seriousness or advancement stage of pathology. According to an aspect of the present invention, the overall duration of the treatment will be evaluated by the attending physician based upon the subject's responses.

According to a preferred aspect, said subject is a human being.

A treatment method too is herein described, comprising at least a step of administering a therapeutically effective amount of an extract or a composition according to any one of the herein described embodiments to a subject requiring it, in particular a subject suffering from any one of the pathologies or disorders mentioned in the present description and claims.

It is declared that the below illustrated examples were performed on informed patients who provided explicit, free and informed consent to the treatments selected according to current legislation.

### EXAMPLES

Some not limiting embodiment examples of the compositions and methods according to the present invention are herein reported by way of example.

### Example 1—Preparation of the Natural Eutectic Solvents (NaDES)

As described in literature (García A, et al. “Extraction of phenolic compounds from virgin olive oil by deep eutectic solvents (DESs).” Food Chem. 2016 Apr. 15; 197(Pt A):554-61. doi: 10.1016/j.foodchem.2015.10.131. Epub 2015 Oct. 27; Paradiso V M, et al. “Towards green analysis of virgin olive oil phenolic compounds: Extraction by a natural deep eutectic solvent and direct spectrophotometric detection.” Food Chem. 2016 Dec. 1; 212:43-7. doi: 10.1016/j.foodchem.2016.05.082; Mouratoglou, et al. (2016). “Novel glycerol-based natural eutectic mixtures and their efficiency in the ultrasound-assisted extraction of antioxidant polyphenols from agri-food waste biomass.” Waste and Biomass Valorization, 7, 1377-1387), the eutectic solvents were prepared by mixing the compounds, according to a precise molar ratio, within a glass container in a water bath and in sonication at high temperature (about 80° C. for at least 90 minutes). Alternatively, it is possible to submit the mixture to magnetic stirring in a water bath (about 80° C. for at least 20 minutes), until obtaining a liquid and homogeneous solution. Based upon the experience one observed that temperature and time required to the formation of NaDES, as well as even colour and viscosity, depend upon the used starting compounds; this led to formulate a different protocol for each used eutectic solvent.

The following eutectic solvents were implemented (mixed in the following molar ratios):
1) Choline: Glycerol (1:1,5) CholGlc
2) Choline: Propylene glycol (1:3) CholProp
3) Betaine: Glycerol (1:2,2) BetGlc
4) Betaine: Propylene glycol (1:3.3) BetProp
5) Proline: Glycerol (1:1,8) ProGlc
6) Proline: Propylene glycol (1:4) ProProp

### Example 2—Process for Extracting the Olive Oil

The olive oil was put in a glass container (becker) under constant magnetic stirring. A NaDES as prepared in Example 1 is mixed with oil in ratio 100:1 to 1:100, preferably 20:1, and left under mechanical stirring at room temperature. The used olive oil preferably is oil coming from cultivar particularly enriched with polyphenols, such as Coratina, Frantoio, Ogliarola. After this process the stirring is stopped and NaDES is separated by simple decantation or through centrifugation at 500 g for 20 minutes.

### Example 3—Determination of the Content of Polyphenols of NaDES

The evaluation of the content of Polyphenols is performed in NaDES phase both by colorimetric method (Folin Ciocalteau) and by chromatographic analysis (UHPLC in reverse phase with spectrophotometric and mass detector). The extract in NaDES is diluted 1:100 in water and 10 uL thereof are put to react with 790 uL of water and 50 uL of reactive Folin-Ciocalteau in presence of 150 uL of 20% Sodium Carbonate. After 2 hours in the dark the absorbance is measured by means of a spectrophometer Hitachi U2000 at the wavelength of 760 nm. The calibration curve is obtained by using as standard a hydroxytyrosol solution, and the content in polyphenols of NaDES is expressed in hydroxytyrosol equivalents per mL of NaDES.

The chromatographic analysis is performed still on 10 uL of NaDES diluted in water according to the above-described modes. The analysis is performed by a UPLC acquity provided with detector photodiode array and mass detector. The determination of the chromatographic profile of the various polyphenols and the quantification of each one thereof, is performed by comparison with standard, by comparing the retention time, the absorption and mass spectrum of each peak.

### Example 4—Preparation of a Serum Based on NaDES Betaine and Propylene Glycol (1:3.3) BetProp

2 portions of NaDES (betaine and propylene glycol (1:3.3)) containing polyphenols+1 portion of almond oil (w/w) were inserted in a turbo emulsifier and the whole was subjected to mixing for 10 minutes. The composition was left to rest for 20 minutes and mixed for further 10 minutes.

### Example 5—Preparation of the Oil Bath

Coratina (70%) oil+water (10%)+surfactants (20%) were inserted in a turbo emulsifier. Then, the mixing phase was started for 10 minutes. One continued with the homogenization phase for 20 minutes.

### Example 6—Cream Preparation

The production provided two phases, a first fatty (lyophilic) phase and a second aqueous (hydrophilic) phase. In the first phase all cream fatty components were inserted in a turbo-emulsifier. These were mixed for 10 minutes. The aqueous portion of the compound was then added and the whole was mixed for 10 minutes. NaDES (betaine and propylene glycol (1:3.3)) containing polyphenols was added and it was subjected to mixing for further 10 minutes. Then one moved to the homogenization phase for 10 minutes. The obtained cream consists by 40% of NaDES.

### Example 7—Treatment of the Atopic Dermatitis (FIG. 1)

A product based on NaDES betaine/propylene glycol, mixed with sweet almond oil in ratio 2:1 (serum described in Example 4), was applied twice a day on the back of the hand of a subject suffering from atopic dermatitis resistant to cortisone-based drugs. The photos illustrated in FIG. 1 were taken immediately before the application, after 10 hours, after 32 hours and after 7 days.

The treatment results show the immediate anti-inflammatory effect which appeared within few hours and the persistent long-term effect, with the lesion resolution.

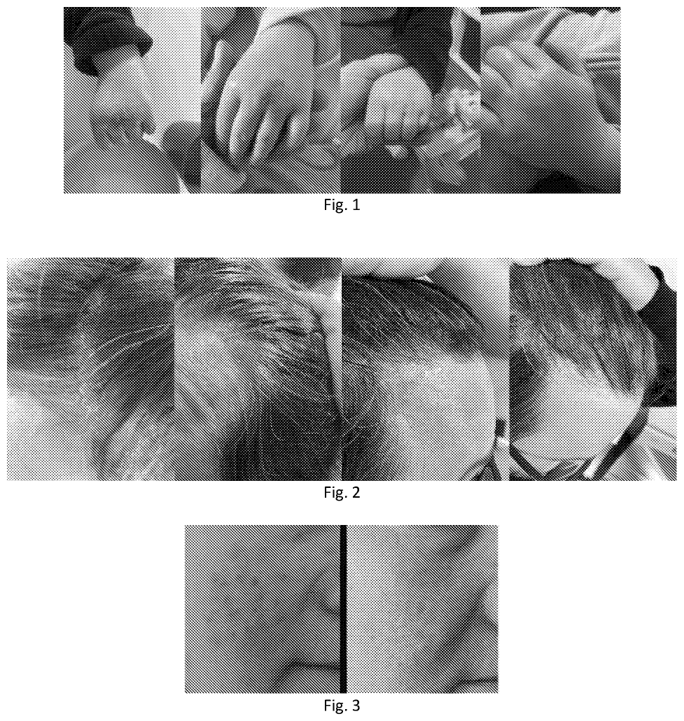

### Example 8—Treatment of Psoriasis (FIG. 2)

A product based on NaDES betaine/propylene glycol mixed with sweet almond oil in ratio 2:1 (serum described in Example 4) was applied twice a day on the scalp of a subject suffering from psoriasis resistant to treatment. Within three days the lesion reduced in size, the inflammation attenuated sensibly and the plaques and scales reduced considerably. Within 6 days the lesion disappeared almost completely.

### Example 9—Treatment of Allergic Dermatitis (FIG. 3)

A product based on NaDES betaine/propylene glycol mixed with sweet almond oil in ratio 2:1 (serum described in Example 4) was mixed in proportion 40:60 to a base cream comprising: water, cetearyl alcohol, Simmondsia chinensis oil, glycerine, Oryza sativa bran oil, squalano, Aloe barbadensis leaf juice, glyceryl stearate, filtered extract of Pseudozyma epicola/Camellia japonica seed oil ferment, olive glyceryl, alkane C13-14, polyglyceryl-4-oleate, cetearyl glycoside, panthenol, perfume, potassium sorbate, sodium benzoate, tocopheryl acetate, xanthan rubber, Mel extract, tetrasodium glutamate diacetate, lactic acid, glucose, sodium hyaluronate, phenoxyethanol, ethylhexylglycerin.

The product was applied on the facial skin suffering from allergic dermatitis. Even in this case the application twice a day reduced the inflammation and the lesions in 24 hours.

### Example 10—Treatment of the Chronic Pain Associated to Psoriatic Arthritis

A product based on NaDES betaine/propylene glycol mixed with sweet almond oil in ratio 2:1 (serum described in Example 4) was applied twice a day on the hand of a subject suffering from psoriasis resistant to treatment with cortisone-based drugs and with reduced mobility. A treatment of 7 days resulted in the disappearance of pain and a restoration of limb mobility.

### Example 11—Stability of Oleocanthal and Oleacein in NaDES or Water (Table 1)

Oleocanthal and oleacein were dissolved at a final concentration equal to 10 mM in water or in NaDES (Betaine/Propylene glycol, Bet/Prop) and subjected to stability test. After 30 days, the losses shown in table are observed.

TABLE 1
Percentage loss of Oleacein and Oleocanthal dissolved in NaDES
or H₂O after 30 days at 25° C. and 40° C.

| | NaDES betaine/propylene | | | |
|---|---|---|---|---|
| | glycol (1:3.3) | | H₂O | |
| | % loss of | | % loss of | % loss of |
| | Oleacein | Oleocanthal | Oleacein | Oleocanthal |
| 25° C. | ~0% | ~0% | 8.1% | 7.5% |
| 40° C. | 4.5% | 1.4% | 38.1% | 32.6% |

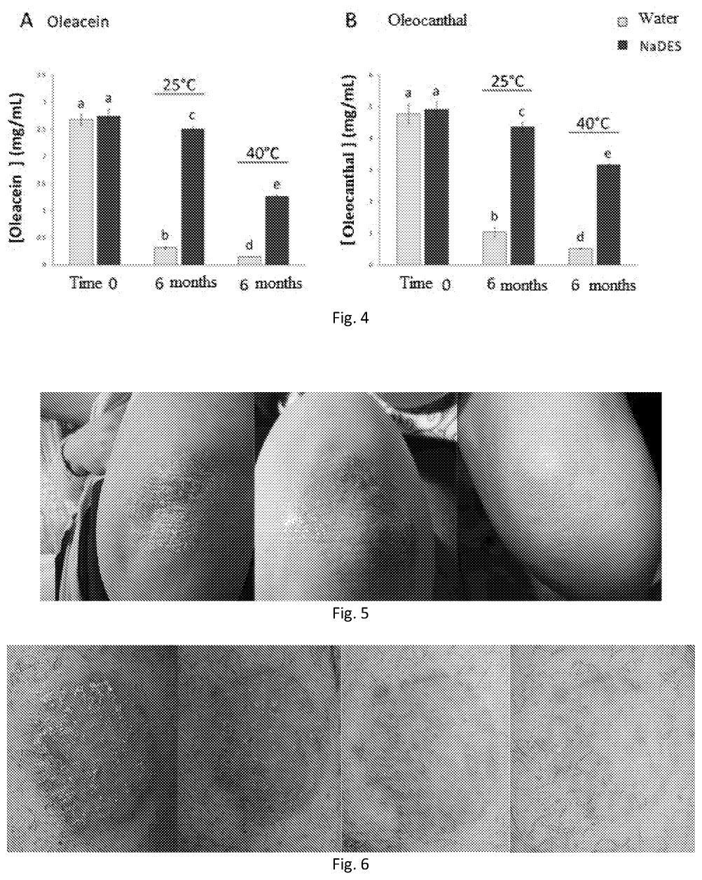

Additional stability tests of oleocanthal and oleacein in water or in NaDES (that is in NaDES mixture comprising betaine/propylene glycol 1:3.3) were performed for 6 months, at different temperatures. The results obtained from the stability tests over time (6 months) of oleacein and oleocanthal, at two different temperatures (25° C. and 40° C. (accelerated stability)), in water and in the formulated product, are shown in FIG. 4. The stability tests show a significantly higher protection of the active principle in the formulated product with respect to the control in water, both at 25° C. and 40° C. In particular, in water, after six months at 25° C., there is a degradation of oleacein and oleocanthal by about 70% and l′80%, respectively. At 40° C. the degradation results to be still more evident: about 90% for oleacein and 80% for oleocanthal. On the contrary, in the product formulated according to the present invention (NaDES betaine/propylene glycol 1:3.3), the degradation of the active principles, with respect to control at time zero, results to be modest at 25° C. and by about 50% and 30% for oleacein and oleocanthal respectively at 40° C.

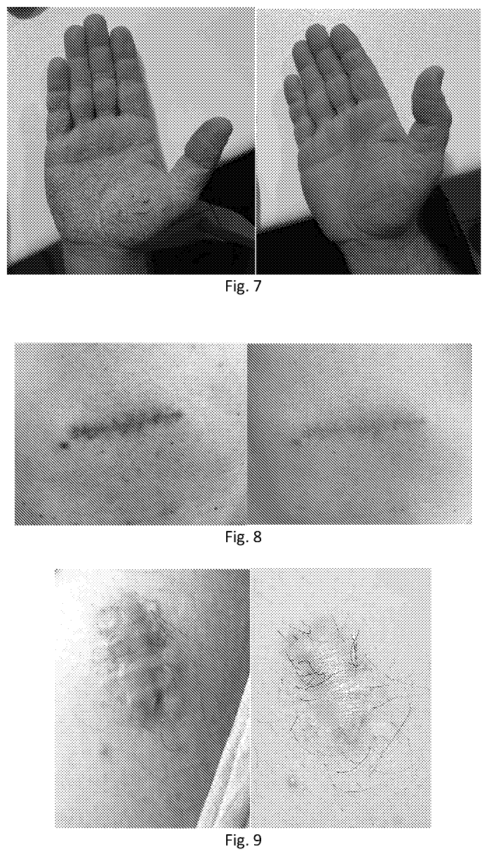

### Example 12—Treatment of Psoriasis (FIGS. 5-7)

Some patients affected by psoriasis were treated for 7/30 days with two applications per day of a serum prepared as described in Example 4 (Polyphenols extracted in NaDES based on betaine/propylene glycol 1:3.3 diluted in almond oil) on permanent lesions resistant to cortisone. As it can be seen from FIGS. 5-7, the application of the product according to the invention led to a significative improvement of the lesions.

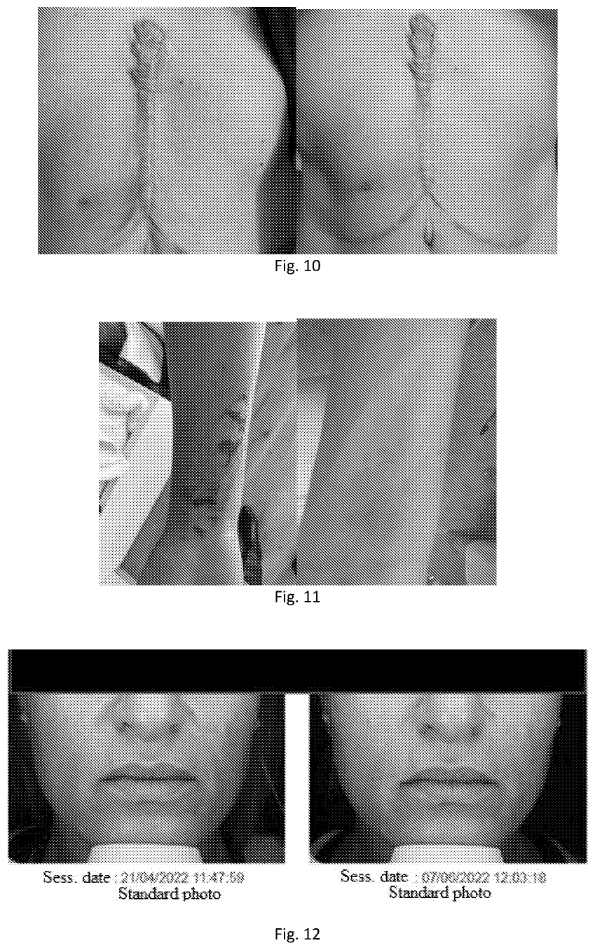

### Example 13—Treatment of Keloids (FIGS. 8-10)

Some patients with keloids or scars following surgical operations were treated for 5/10 days with two applications per day of a serum prepared as described in Example 4 (Polyphenols extracted in NaDES based on betaine/propylene glycol 1:3.3, in almond oil) on aged lesions (going back to previous months or years). As it can be seen from FIGS. 8-10, the application of the product according to the invention led to a significative improvement of the lesions.

### Example 14—Treatment of Burns (FIG. 11)

Some patients with burns or scalding were treated for 1/10 days with two applications per day of a serum prepared as described in Example 4 (Polyphenols extracted in NaDES betaine/propylene glycol based on 1:3.3 diluted in almond oil). As it can be seen from FIG. 12, the application of the product according to the invention led to a significative improvement of the lesions.

### Example 15—Anti-Aging Activity

The application of the product according to the present invention in form of cream prepared as described in Example 6 (Polyphenols extracted in NaDES betaine/propylene glycol based on 1:3.3, in almond oil) on the skin of a healthy subject for 45 days showed a significative anti-aging activity.

1. An extract in eutectic solvent of olive oil polyphenols.

2. The extract according to claim 1, wherein said eutectic solvent is a natural deep eutectic solvent.

3. The extract according to claim 1, wherein said eutectic solvent comprises at least a hydrogen bond acceptor compound selected from the group consisting of choline, betaine, proline and carnitine, and at least a hydrogen bond donor compound selected from the group consisting of propylene glycol, 1,3-propanediol, glycerol and ethylene glycol.

4. The extract according to claim 1, wherein said eutectic solvent is a natural deep eutectic solvent comprising propylene glycol and betaine.

5. The extract according to claim 1, wherein said eutectic solvent is a natural deep eutectic solvent comprising 1,3-propanediol and betaine.

6. The extract according to claim 1, wherein said polyphenols comprise oleocanthal and/or oleacein.

7. The extract according to claim 1, wherein the extract is present in a composition comprising the extract and at least a carrier or excipient.

8. The extract according to claim 7, wherein the composition is in a form selected from serum, cream, spray, gel, solution, suspension, oil bath, or shampoo.

9. The extract according to claim 1, wherein the extract is coated on or is soaking a medical device.

10. A method of using the extract of claim 1 as a medicament, the method comprising topically administering th extract to a subject.

11. The method according to claim 10, wherein the administering treats or assists in the treatment of a pathology or disorder of the skin.

12. The method according to claim 11, wherein said pathology or disorder of the skin is selected from atopic dermatitis, psoriasis, keloids, seborrheic dermatitis, acne, rosacea, allergic dermatitis, and skin redness.

13. The method according to claim 10, wherein the administering prevents and/or treats or assists in the treatment of a skin lesion.

14. The method according to claim 13, wherein said skin lesion is selected from scalding, a burn, a scald, and an insect stings.

15. The method according to claim 10, wherein the administering prevents and/or treats skin damage or a skin lesion derived or caused by radiation.

16. The method according to claim 15, wherein said skin damage or lesions derived or caused by radiation is derived or caused by exposure to sunlight, radiotherapy and/or radio-chemotherapy.

17. The method according to claim 10, wherein the administering promotes skin healing, prevents and/or treats imperfections of skin of the subject, and/or reduces and/or removes wrinkle and/or ageing of the skin of a subject.

18-19. (canceled)

20. A process for preparing an extract of olive oil polyphenols comprising the following steps of:
(i) providing an olive oil comprising said polyphenols;
(ii) putting in contact said olive oil with at least one eutectic solvent for a period of time sufficient to allow the extraction of said polyphenols in said at least one eutectic solvent;
(iii) separating the extract of polyphenols in eutectic solvent obtained in step (ii) from olive oil.

21. The process according to claim 20, wherein said eutectic solvent is a natural deep eutectic solvent comprising propylene glycol and betaine.

22. The process according to claim 20, wherein said eutectic solvent is a natural deep eutectic solvent comprising 1,3-propanediol and betaine.

23-25. (canceled)
